(19) United States
(12) Patent Application Publication
(10) Pub. No.: US 2019/0380939 A1
(43) Pub. Date: Dec. 19, 2019

(54) COSMETIC PREPARATION WITH EXTRAORDINARY TEXTURE FOR TEMPORARILY SHAPING KERATINIC FIBRES

(71) Applicant: Henkel AG & Co. KGaA, Duesseldorf (DE)

(72) Inventors: MARCUS NOLL, Quickborn (DE); LARA STEGMANN, Hamburg (DE); ARNE JUNGE, Hamburg (DE)

(73) Assignee: Henkel AG & Co. KGaA, Duesseldorf (DE)

(21) Appl. No.: 16/418,486

(22) Filed: May 21, 2019

(30) Foreign Application Priority Data
Jun. 13, 2018 (DE) 10 2018 114 107.6

(51) Int. Cl. A61K 8/58; A61K 8/891; A61K 8/73; A61Q 5/06; A61K 8/25

(52) U.S. Cl. CPC A61K 8/585; A61K 8/891; A61K 2800/412; A61Q 5/06; A61K 8/25; A61K 8/731

(57) ABSTRACT

A cosmetic preparation for temporarily shaping keratinic fibers comprises a hydrophobically modified silica, a particulate cellulose and a polyorganosiloxane.

### CROSS-REFERENCE TO RELATED APPLICATION

This application claims priority to German Patent Application No. 10 2018 114 107.6, filed Jun. 13, 2018, which is incorporated herein by reference in its entirety.

### TECHNICAL FIELD

The present disclosure relates to cosmetic preparations for temporarily shaping keratinic fibers, comprising hydrophobically modified silica. Furthermore, the present disclosure relates to the use of the cosmetic preparation as contemplated herein.

### BACKGROUND

The term “keratinic fibers” should principally be understood to mean any animal hair, for example wool, horsehair, angora hair, fur, feathers and products or textiles produced therefrom. In particular, however, it should be understood to mean human hair.

Cosmetic agents which serve to permanently or temporarily style the hair have an important role in cosmetics. Temporary styling, which should provide a good hold without compromising the healthy appearance of the hair, such as its shine, for example, can be obtained by using hair sprays, hair waxes, hair gels, hair mousses etc, for example. The cosmetic agents are blended as a function of these forms of applications.

An important property of an agent for temporarily shaping keratinic fibers is that the treated fibers have to be endowed with as strong a hold as possible in the shape which is produced. When the keratinic fibers are human hair, this is also known as a strong styling hold or a high degree of hold of the cosmetic agent.

In addition to the essential requirement for all styling agents to endow the treated hair with the desired hold, it is currently more important that in its point-of-sale format, the product should have an appealing external appearance, or an interesting consistency. Cosmetic products with novel, exciting textures are very popular. Extraordinary product textures appeal to specific classes of consumers and therefore lead to a decision to purchase.

An interesting external appearance with cosmetic products must always, however, be accompanied by appropriate or even excellent product properties. The consumers want a natural look which looks neither artificial nor overstyled. Furthermore, the hair should not be made heavier by the styling agent. Consumers expect a whole series of further excellent styling properties.

Numerous cosmetic agents for providing temporary styling are known in the prior art. However, there is still a need for cosmetic preparations which provide extraordinary textures as regards the appearance of the product in harmony with excellent styling properties endowed on the hair by the product.

### BRIEF SUMMARY

In accordance with an exemplary embodiment, a cosmetic preparation for temporarily shaping keratinic fibers is provided. The cosmetic preparation comprises a hydrophobically modified silica, a particulate cellulose, and a polyorganosiloxane.

In accordance with another exemplary embodiment, a method for temporarily shaping keratinic fibers is provided. The method comprises applying to the keratinic fibers a cosmetic preparation comprising a hydrophobically modified silica, a particulate cellulose, and a polyorganosiloxane. The method further includes bringing the keratinic fibers into a desired shape.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The fundamental objective of the present disclosure lies in the provision of a cosmetic preparation which exhibits an extraordinary texture as regards its appearance, wherein the styling properties endowed on the hair by the preparation are of a high standard.

The fundamental objective of the present disclosure is achieved by employing the subject matter of claim 1. Thus, in a first aspect, the present disclosure provides a cosmetic preparation for temporarily shaping keratinic fibers, in particular human hair, which comprises a hydrophobically modified silica, a particulate cellulose and a polyorganosiloxane.

As a first component, the cosmetic preparation contains a hydrophobically modified silica. The term “hydrophobically modified silica” as used in the context of the present disclosure should be understood to mean any amorphous or partially amorphous silicon dioxide with silanol groups on its surface which have been partially or completely chemically reacted so that after the reaction, at least a portion of the surface has been provided with an organic residue. Hydrophobically modified silicas are used in many technical fields and a large variety have been described in the prior art.

The hydrophobically modified silica has a plurality of functions in the cosmetic preparation: it has a significant influence on the styling effect. If the proportion of hydrophobically modified silica in the composition increases, then network-like structures can form by hydrophobic interactions between the particles of the hydrophobically modified silica. When the components of the medium for the cosmetic preparation evaporate off, i.e. after applying the cosmetic preparation to the hair, the relative proportion of the hydrophobically modified silica on the hair increases. The hydrophobically modified silica is a component of a film which strengthens the hair. In this manner, the cosmetic preparation can produce a styling effect.

Furthermore, the hydrophobically modified silica varies the rheological behavior of the cosmetic preparation. Hydrophobically modified silicas are in fact not wetted by water, but rather by the components which form a hydrophobic medium. In a hydrophobic medium, hydrophobically modified silicas constitute a suitable additive for controlling the rheology. Alternatively, hydrophobically modified silicas may be used in aqueous media in order to control the rheology when suitable binders are employed.

In accordance with a preferred embodiment of the present disclosure, the hydrophobically modified silica in the cosmetic preparation is a silylation product formed from a silica and an organosilicon compound. In accordance with a further preferred embodiment, the silica is a pyrogenic silica. Preferably, hydrophobically modified silicas which are suitable for use as contemplated herein contain hydrophobic groups such as (CH₃)₃Si—O—, (—Si(CH₃)₂O—)ₙ, (H₃C)₂Si(O)₂— and C₇H₁₈Si(O)₃— at their surface. Alkyl-modified hydrophobic pyrogenic silicas that are commercially obtainable, for example under the trade names Aerosil™R (Evonik)[[.]], Aerosil™R202, Aerosil™R805, Aerosil™R812, Aerosil™R972, Aerosil™R974 and Aerosil™R976, as well as the commercially available products HDK™H2000, HDK™H2050 and HDK™H3004 (Wacker), are particularly preferred.

Cosmetic preparations comprising a hydrophobically modified silica in which the organosilicon compound used is an alkylsiloxane or a silazane are particularly preferred, wherein more particularly preferably, the organosilicon compound is an alkylsilazane, in particular hexamethyldisilazane.

In accordance with a preferred embodiment, the hydrophobically modified silica is contained in the cosmetic preparation in a quantity of from about 2 to about 20% by weight, preferably of from about 3 to about 10% by weight, more preferably of from about 4 to about 8% by weight, with respect to the total weight of the cosmetic preparation.

The cosmetic preparation contains a particulate cellulose as the second component. In accordance with a preferred embodiment, the particulate cellulose is a microcrystalline cellulose.

Microcrystalline cellulose is obtained from lignified plant parts. Cellulose is the main component of plant cell walls with a proportion by weight of approximately 50%, and is thus the most abundant polysaccharide. It is not branched and consists of several hundred to many thousands of β-D-glucose molecules (β-1,4-glycoside bonding). The non-crystalline cellulose fractions in the lignified plant parts are separated out using an acidic medium at temperatures of over about 100° C. The microcrystalline cellulose may occasionally be carboxylated. Microcrystalline cellulose is an excellent rheology modifier for use in cosmetic agents. The feel of the cosmetic preparation can readily be controlled by the proportions and grain size thereof.

In accordance with a preferred embodiment of the present disclosure, the particulate cellulose in the cosmetic preparation is a microcrystalline cellulose with a mean particle size of from about 2 to about 50 μm, preferably of from about 3 to about 25 μm, more preferably of from about 4 to about 15 μm. The mean particle size is an arithmetic mean which is determined by measuring the powder under an optical microscope, wherein for each particle, the longest cross section is taken in order to produce the arithmetic mean, wherein a sufficient quantity of particles is measured to form the arithmetic mean.

In accordance with a further preferred embodiment of the present disclosure, the particulate cellulose in the cosmetic preparation is a microcrystalline cellulose with a mass density of from about 100 to about 200 g/L, preferably of from about 120 to about 195 g/L, more preferably of from about 140 to about 190 g/L. The mass density has an influence on the ability of the microcrystalline cellulose to be worked into the mass in order to produce the cosmetic preparation.

Furthermore, in accordance with a further preferred embodiment, the cosmetic preparation contains the microcrystalline cellulose in a quantity of from about 15 to about 60% by weight, preferably of from about 20 to about 45% by weight, more preferably of from about 30 to about 40% by weight, with respect to the total weight of the cosmetic preparation.

The properties of the microcrystalline cellulose and its quantity have a very significant influence on the external appearance of the cosmetic preparation, in particular the product texture and the consistency. The fundamental objectives of the present disclosure are achieved in a particularly good manner within the above limits: the cosmetic preparations as contemplated herein are distinguished by a unique consistency. The appearance is similar to that of an opaque gel, however the mass is substantially more stable in shape than conventional styling gels. Nevertheless, the mass is flexible and significantly softer than conventional hair waxes, for example. If the mass is rubbed between the fingers, it feels pleasantly dry and is not sticky. The particulate components contribute to an unusual external appearance.

The cosmetic preparation contains a polyorganosiloxane as the third component of the present disclosure. The polyorganosiloxanes used in the cosmetic preparations essentially act as a support for the other components of the cosmetic preparation. The suitable polyorganosiloxanes compatibilize the hydrophobically modified silica and the particulate cellulose. The further advantage with using polyorganosiloxanes is that it endows the hair with shine and smoothness. A further advantage which is associated with the polyorganosiloxanes is that other solvents do not necessarily have to be added.

In accordance with a preferred embodiment, the polyorganosiloxane of the cosmetic preparation is a linear, cyclic or elastomeric polydimethylsiloxane, wherein preferably, the linear or elastomeric polydimethylsiloxane with formula (CH₃)₃SiO[SiO(CH₃)₂]ₙSi(CH₃)₃ in which n=from about 4 to about 100, more preferably n=from about 5 to about 50, most preferably n=from about 10 to about 25, is sufficient, or wherein preferably, the cyclic polydimethylsiloxane has the basic structure [—Si—(CH₃)₂O—]ₙ in which n=from about 4 to about 12, more preferably n=from about 4 to about 8, most preferably n=about 5. In this regard, cyclotetrasiloxane, cyclopentasiloxane, cyclohexasiloxane, cycloheptasiloxane or cyclooctasiloxane, in particular cyclopentasiloxane, are preferably used.

In accordance with a preferred embodiment, the viscosity of the polyorganosiloxane in the cosmetic preparation is from about 1 to about 100 cSt, preferably from about 2 to about 75 cSt, more preferably from about 5 to about 50 cSt. In order to determine the viscosity of the polyorganosiloxane, a falling sphere viscometer is used. Its viscosity can be determined with the assistance thereof and by following DIN 53015. The manufacturer's data regarding the viscosity of the polyorganosiloxanes can be used in order to simplify the determination as to which polyorganosiloxanes can be used in the cosmetic preparation as contemplated herein. The polyorganosiloxanes used are all commercially available and are usually classified according to their viscosities, measured in Stokes.

The term “elastomeric polydimethylsiloxane” should be understood to mean a polymeric polydimethylsiloxane with a glass transition temperature which is below the temperature of the intended use, for example below 0° C. Preferably, the elastomeric polyorganosiloxane is a cross-linked polysiloxane which is obtained by reaction of a polysiloxane, which comprises at least one hydrogen atom bonded directly to a silicon atom, with an alpha, omega-alkyldiene containing 5 to 24 carbon atoms. Appropriate silicone elastomers may be produced in accordance with the teaching of U.S. Pat. No. 5,654,362 A.

In a further preferred embodiment of the present disclosure, the polyorganosiloxane is used in the form of a blend of a cross-linked elastomeric polyorganosiloxane with the INCI name dimethicone crosspolymer and cyclopentasiloxane. Appropriate mixtures are commercially available and are marketed, for example by Dow Corning under the trade name Dow Corning™ 9040 Silicone Elastomer Blend and Dow Corning™ 9045 Silicone Elastomer Blend. The use of Dow Corning™ 9040 Silicone Elastomer Blend is more particularly preferred.

Preferably, the cosmetic preparations as contemplated herein contain polyorganosiloxanes with formula (I)

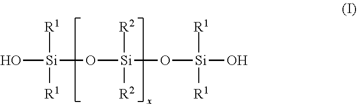

### or (II)

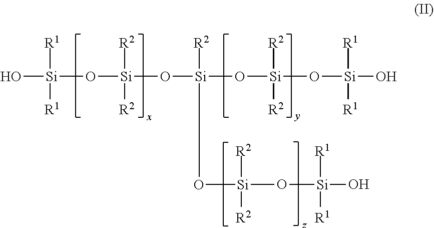

wherein, in formulae (I) and (II):

R¹ represents hydrogen, a methyl residue, a C2 to C30 linear, branched or unsaturated hydrocarbon residue, a phenyl residue and/or an aryl residue,

R² represents hydrogen, a methyl residue, a C2 to C30 linear, branched or unsaturated hydrocarbon residue, a phenyl residue and/or an aryl residue, and

x, y and z, respectively independently of each other, represent a whole number from 0 to about 20000.

Non-limiting examples of the residues represented by R¹ and R² include alkyl residues such as methyl, ethyl, propyl, isopropyl, butyl, isobutyl, pentyl, isopentyl, neopentyl, amyl, isoamyl, hexyl, isohexyl and decyl, alkenyl residues such as vinyl, vinyl halide, alkylvinyl, allyl, allyl halide, alkylallyl, phenyl residues and benzyl residues. Preferably, R¹ and R², respectively independently of each other, represent an alkyl residue which contains 1 to 6 carbon atoms; particularly preferably, R¹ and R² represent methyl. Preferably, x, y and z, respectively independently of each other, represent whole numbers from 0 to about 10000.

In accordance with a preferred embodiment of the present disclosure, the cosmetic preparation contains the polyorganosiloxane in a quantity of from about 20 to about 80% by weight, preferably of from about 40 to about 70% by weight, more preferably of from about 50 to about 60% by weight, with respect to the total weight of the cosmetic preparation. The advantageous effects exert the best influence within these ranges of the quantities. The type and quantity of the polyorganosiloxane influence the outcome as regards shine and smoothness.

Preferably, the cosmetic preparations are essentially anhydrous. In particular, the person skilled in the art will understand from this that no water is added. Because of the fact that raw materials in general, and in respect of the present disclosure the silica as well as the microcrystalline cellulose in particular, are not entirely free from water, the cosmetic preparation as contemplated herein will always contain a small quantity of water. Furthermore, products always absorb a certain amount of moisture. Nevertheless, a preferred cosmetic preparation will essentially be free from water. Preferably, the cosmetic preparation comprises above 0 to about 5% by weight of water, preferably above 0 to about 3% by weight, most preferably above 0 to about 1% by weight, with respect to the total weight of the cosmetic preparation.

Particularly preferred cosmetic preparations essentially consist of the hydrophobically modified silica, the microcrystalline cellulose and the polyorganosiloxane. The three components achieve the fundamental objective of the present disclosure. An advantage of the present disclosure lies in the lack of complexity of the mixture which forms the cosmetic preparation of the present disclosure.

As discussed above, the polyorganosiloxanes act as a support in the cosmetic preparation as contemplated herein, so that other solvents can be dispensed with. The cosmetic preparations may contain alcohols, however. Ethanol and isopropanol are advantageous alcohols in this regard because of their commercial availability.

Organic solvents or a mixture of solvents with a boiling point of below about 400° C. in a quantity of from about 0.1 to about 15% by weight, preferably of from about 1 to about 10% by weight with respect to the total weight of the cosmetic preparation, may be used as additional co-solvents. Particularly suitable additional co-solvents are unbranched or branched hydrocarbons such as pentane, hexane, isopentane, and cyclic hydrocarbons such as cyclopentane and cyclohexane. Other particularly preferred solvents are glycerin, ethylene glycol, propylene glycol and phenoxyethanol in a quantity of up to about 15% by weight with respect to the total weight of the cosmetic preparation.

The cosmetic preparation may furthermore contain adjuvants, agents and additives which are known and frequently used in styling agents. As an example, the cosmetic preparation as contemplated herein may furthermore contain at least one emulsifier or a surfactant. In this regard, however, it should be noted that larger quantities of emulsifiers or surfactants may have a negative effect on the desired consistency of the cosmetic preparation. The type and quantity of these components should therefore be carefully selected. Preferably, emulsifiers and surfactants are dispensed with.

In principle, any anionic, ampholytic, cationic and non-ionic surface-active compounds which are suitable for use on the human body may be considered as emulsifiers. In this regard, the group of ampholytic surface-active compounds includes zwitterionic surface-active compounds and amphoteric compounds. The use of anionic and non-ionic surface-active compounds when used in the cosmetic preparation is preferred in the present disclosure.

Anionic surface-active compounds are exemplified by an anionic group such as, for example, a carboxylate, sulfate, sulfonate or phosphate group, which makes the compound water-soluble, and a lipophilic alkyl group containing approximately 10 to about 22 C atoms. In addition, the molecule may contain glycol or polyglycolether groups, ester, ether and amide groups, as well as hydroxyl groups. Non-ionic surface-active compounds contain, as the hydrophilic group, a polyol group, a polyalkyleneglycolether group or a combination of polyol and polyglycolether groups, for example. Preferred non-ionogenic surface-active compounds are the addition products of alkylene oxide, in particular ethylene oxide, with fatty alcohols and fatty acids. Particular examples of cationic surface-active compounds are quaternary ammonium compounds. Ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride are preferred. Furthermore, the very readily degradable quaternary ester compounds such as those sold under the trade name Dehyquart™, as well as quaternized protein hydrolysates and silicone compounds, may be used as contemplated herein.

If emulsifiers are used, they are preferably used in quantities of from about 0.1 to about 10% by weight, particularly preferably in quantities of from about 0.5 to about 5% by weight and more particularly preferably in quantities of from about 0.7 to about 3% by weight, wherein the quantities given are respectively with respect to the total quantity of the cosmetic preparation.

The adjuvants or additives which are conventionally used in cosmetic preparations also usually include nourishing products. Because the cosmetic preparation already contains various silicones, however, which also have nourishing properties, as a rule, the addition of further nourishing products can be dispensed with.

More particularly preferred cosmetic preparations as contemplated herein include at least one of the following embodiments A) to H):

### A)

Cosmetic preparation for temporarily shaping keratinic fibers, in particular human hair, comprising a hydrophobically modified silica, which is a silylation product formed from a pyrogenic silica and an organosilicon compound, a microcrystalline cellulose and a polyorganosiloxane.

### B)

Cosmetic preparation for temporarily shaping keratinic fibers, in particular human hair, comprising a hydrophobically modified silica, which is a silylation product formed from a pyrogenic silica and an organosilicon compound, a microcrystalline cellulose and a polyorganosiloxane, wherein the microcrystalline cellulose has a mean particle size of from about 2 to about 50 μm, preferably of from about 3 to about 25 μm, more preferably of from about 4 to about 15 μm.

### C)

Cosmetic preparation for temporarily shaping keratinic fibers, in particular human hair, comprising a hydrophobically modified silica, which is a silylation product formed from a pyrogenic silica and an organosilicon compound, a microcrystalline cellulose and a polyorganosiloxane which is a linear or elastomeric polydimethylsiloxane with formula (CH₃)₃SiO[SiO(CH₃)₂]ₙSi(CH₃)₃ in which n=from about 4 to about 100, preferably n=from about 5 to about 50, most preferably n=from about 10 to about 25, or which is a cyclic polydimethylsiloxane with the basic structure [—Si—(CH₃)₂O—]ₙ in which n=from about 4 to about 12, more preferably n=from about 4 to about 8, most preferably n=about 5.

### D)

Cosmetic preparation for temporarily shaping keratinic fibers, in particular human hair, comprising a hydrophobically modified silica, which is a silylation product formed from a pyrogenic silica and an organosilicon compound, a microcrystalline cellulose and a polyorganosiloxane, which is a mixture of a cross-linked elastomeric polyorganosiloxane with the INCI name dimethicone crosspolymer and a cyclopentasiloxane.

### E)

Cosmetic preparation for temporarily shaping keratinic fibers, in particular human hair, consisting of a hydrophobically modified silica, which is a silylation product formed from a pyrogenic silica and an organosilicon compound, a microcrystalline cellulose and a polyorganosiloxane, wherein the microcrystalline cellulose has a mean particle size of from about 2 to about 50 μm, preferably of from about 3 to about 25 μm, more preferably of from about 4 to about 15 μm.

### F)

Cosmetic preparation for temporarily shaping keratinic fibers, in particular human hair, comprising a hydrophobically modified silica, which is a silylation product formed from a pyrogenic silica and an organosilicon compound, a microcrystalline cellulose and a polyorganosiloxane, wherein the microcrystalline cellulose has a mean particle size of from about 2 to about 50 μm, preferably of from about 3 to about 25 μm, more preferably of from about 4 to about 15 μm, wherein the cosmetic preparation is anhydrous, preferably wherein the cosmetic preparation has a water content of above 0 to about 5% by weight, more preferably of above 0 to about 3% by weight, most preferably of above 0 to about 1% by weight, with respect to the total weight of the cosmetic preparation.

### G)

Cosmetic preparation for temporarily shaping keratinic fibers, in particular human hair, comprising a hydrophobically modified silica, which is a silylation product formed from a pyrogenic silica and an organosilicon compound, a microcrystalline cellulose and a polyorganosiloxane, wherein the microcrystalline cellulose has a mean particle size of from about 2 to about 50 μm, preferably of from about 3 to about 25 μm, more preferably of from about 4 to about 15 μm, and wherein the hydrophobically modified silica is used in the cosmetic preparation in a quantity of from about 2 to about 20% by weight, preferably of from about 3 to about 10% by weight, more preferably of from about 4 to about 8% by weight; the microcrystalline cellulose is used in the cosmetic preparation in a quantity of from about 15 to about 60% by weight, preferably of from about 20 to about 45% by weight, more preferably of from about 30 to about 40% by weight, and the polyorganosiloxane is used in the cosmetic preparation in a quantity of from about 20 to about 80% by weight, preferably of from about 40 to about 70% by weight, more preferably of from about 50 to about 60% by weight, respectively with respect to the total weight of the cosmetic preparation.

### H)

Cosmetic preparation for temporarily shaping keratinic fibers, in particular human hair, comprising a hydrophobically modified silica, which is a silylation product formed from a pyrogenic silica and an organosilicon compound, a microcrystalline cellulose and a polyorganosiloxane, wherein the microcrystalline cellulose has a mean particle size of from about 2 to about 15 μm, and wherein the hydrophobically modified silica is used in the cosmetic preparation in a quantity of from about 2 to about 10% by weight; the microcrystalline cellulose is used in the cosmetic preparation in a quantity of from about 30 to about 45% by weight and the polyorganosiloxane is used in the cosmetic preparation in a quantity of from about 40 to about 66% by weight, respectively with respect to the total weight of the cosmetic preparation, wherein the cosmetic preparation is essentially anhydrous.

The fundamental objective of the present disclosure is furthermore achieved by the subject matter of claim 10. In a second aspect, therefore, the present disclosure concerns a method of temporarily shaping keratinic fibers by applying the cosmetic preparation of the first aspect of the present disclosure and brining the keratinic fibers into a desired shape.

Features relating to preferred embodiments of the first aspect of the present disclosure which are only described above in respect thereof are clearly also applicable to the second aspect as features of preferred embodiments.

The examples below are intended to illustrate the subject matter of the present disclosure without in any way limiting it.

### EXAMPLES

The following preparations as contemplated herein could be manufactured:

### Styling Clay 1

| | | | Quantity |
|---|---|---|---|
| | | | (as weight |
| Trade name | INCI | Manufacturer | %) |
| Vivapur CS | Microcrystalline | J. Rettenmaier & | 33.3 |
| | cellulose | Söhne GmbH & | |
| | | Co. KG | |
| Dow Corning 200 | Dimethicone | Dow Chemicals | 59.3 |
| 50 cs | | | |
| Aerosil R 812 S | Silica silylate | Evonik Degussa AG | 7.4 |
| <Total> | | | 100 |

### Styling Clay 2

| | | | Quantity |
|---|---|---|---|
| | | | (as weight |
| Trade name | INCI | Manufacturer | %) |
| Vivapur CS | Microcrystalline | J. Rettenmaier & | 40 |
| | Cellulose | Söhne GmbH & | |
| | | Co. KG | |
| Dow Corning 200 | Dimethicone | Dow Chemicals | 50 |
| 5 cs | | | |
| Aerosil R 812 S | Silica silylate | Evonik Degussa AG | 10 |
| <Total> | | | 100 |

### Styling Clay 3

| | | | Quantity |
|---|---|---|---|
| | | | (as weight |
| Trade name | INCI | Manufacturer | %) |
| Vivapur CS | Microcrystalline | J. Rettenmaier & | 40 |
| | cellulose | Söhne GmbH & | |
| | | Co. KG | |
| Dow Corning 9040 | Dimethicone | Dow Chemicals | 55 |
| | crosspolymer | | |
| Aerosil R 812 S | Silica silylate | Evonik Degussa AG | 5 |
| <Total> | | | 100 |

### Styling Clay 4

| | | | Quantity |
|---|---|---|---|
| | | | (as weight |
| Trade name | INCI | Manufacturer | %) |
| Vivapur CS | Microcrystalline | J. Rettenmaier & | 30 |
| | cellulose | Söhne GmbH & | |
| | | Co. KG | |
| Cyclopentasiloxane | Cyclomethicone | Dow Chemicals | 60 |
| Aerosil R 812 S | Silica silylate | Evonik Degussa AG | 10 |
| <Total> | | | 100 |

In order to manufacture the styling clays 1 to 4, initially, polyorganosiloxane was provided and the hydrophobically modified silica was added first, with slow stirring. Next, the microcrystalline cellulose was added. Stirring was continued until a homogeneous mass was obtained.

The styling agents were exemplified by a unique consistency which was reminiscent of jelly. The mass was flexible and left a pleasant dry, non-sticky feel on the skin.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A cosmetic preparation for temporarily shaping keratinic fibers, comprising:
a hydrophobically modified silica,
a particulate cellulose, and
a polyorganosiloxane.

2. The cosmetic preparation according to claim 1, wherein the hydrophobically modified silica comprises a silylation product formed from a silica and an organosilicon compound.

3. The cosmetic preparation according to claim 2, wherein the organosilicon compound comprises an alkylsiloxane or a silazane.

4. The cosmetic preparation according to claim 1, wherein the hydrophobically modified silica is present in the cosmetic preparation in a quantity of from about 2 to about 20% by weight with respect to the total weight of the cosmetic preparation.

5. The cosmetic preparation according to claim 1, wherein the particulate cellulose is a microcrystalline cellulose having a mean particle size of from about 2 to about 50 μm, having a mass density of from about 100 to about 200 g/L, or a combination thereof.

6. The cosmetic preparation according to claim 1, wherein the particulate cellulose is present in the cosmetic preparation in a quantity of from about 15 to about 60% by weight with respect to the total weight of the cosmetic preparation.

7. The cosmetic preparation according to claim 1, wherein the polyorganosiloxane comprises a linear, cyclic or elastomeric polydimethylsiloxane, wherein the linear or elastomeric polydimethylsiloxane has the formula (CH₃)₃SiO[SiO(CH₃)₂]ₙSi(CH₃)₃ in which n=from about 4 to about 100, or wherein the cyclic polydimethylsiloxane has the structure [—Si—(CH₃)₂O—]ₙ in which n=4 to 12.

8. The cosmetic preparation according to claim 1, wherein the polyorganosiloxane is present in the cosmetic preparation in a quantity of from about 20 to about 80% by weight with respect to the total weight of the cosmetic preparation.

9. The cosmetic preparation according to claim 1, wherein the cosmetic preparation has a water content of above 0 to about 5% by weight of water, with respect to the total weight of the cosmetic preparation.

10. A method for temporarily shaping keratinic fibers, the method comprising the steps of:
applying to the keratinic fibers a cosmetic preparation comprising:
a hydrophobically modified silica,
a particulate cellulose, and
a polyorganosiloxane, and
bringing the keratinic fibers into a desired shape.

11. The method according to claim 10, wherein the hydrophobically modified silica comprises a silylation product formed from a silica and an organosilicon compound.

12. The method according to claim 11, wherein the organosilicon compound comprises an alkylsiloxane or a silazane.

13. The method according to claim 10, wherein the hydrophobically modified silica is present in the cosmetic preparation in a quantity of from about 2 to about 20% by weight with respect to the total weight of the cosmetic preparation.

14. The method according to claim 10, wherein the particulate cellulose is a microcrystalline cellulose having a mean particle size of from about 2 to about 50 μm, having a mass density of from about 100 to about 200 g/L, or a combination thereof.

15. The method according to claim 10, wherein the particulate cellulose is present in the cosmetic preparation in a quantity of from about 15 to about 60% by weight with respect to the total weight of the cosmetic preparation.

16. The method according to claim 10, wherein the polyorganosiloxane comprises a linear, cyclic or elastomeric polydimethylsiloxane, wherein the linear or elastomeric polydimethylsiloxane has the formula (CH₃)₃SiO[SiO(CH₃)₂]ₙSi(CH₃)₃ in which n=from about 4 to about 100, or wherein the cyclic polydimethylsiloxane has the formula [—Si—(CH₃)₂O—]ₙ in which n=4 to 12.

17. The cosmetic preparation according to claim 1, wherein the hydrophobically modified silica comprises a pyrogenic silica.

18. The cosmetic preparation according to claim 1, wherein the organosilicon compound comprises hexamethyldisilazane.

19. The cosmetic preparation according to claim 1, wherein the viscosity of the polyorganosiloxane is from about 1 to 100 cSt.

20. The cosmetic preparation according to claim 1, wherein the cosmetic preparation consists essentially of:
a hydrophobically modified silica,
a particulate cellulose, and
a polyorganosiloxane.
